(19) United States
(12) Patent Application Publication
(10) Pub. No.: US 2022/0386975 A1
(43) Pub. Date: Dec. 8, 2022

(54) X-RAY APPARATUS

(71) Applicant: IPG PHOTONICS CORPORATION, OXFORD, MA (US)

(72) Inventors: Alexandre EFIMOV, Nashua, NH (US); Dan PERLOV, Sudbury, MA (US); Edward TZIDILKOVSKI, Chelmsford, MA (US)

(73) Assignee: IPG PHOTONICS CORPORATION, OXFORD, MA (US)

(21) Appl. No.: 17/791,583

(22) PCT Filed: Jan. 11, 2021
(86) PCT No.: PCT/US21/12997

§ 371 Date: Jul. 8, 2022

Related U.S. Application Data
(60) Provisional application No. 62/959,353, filed on Jan. 10, 2020.

(51) Int. Cl. A61B 6/00; G21K 1/06

(52) U.S. Cl. CPC A61B 6/484; G21K 1/06; G21K 2201/062

(57) ABSTRACT

An X-ray optical system incorporates a refractometer, interferometer, spectrometer, diffractometer or imaging device for analyzing a sample. The X-ray optical system is configured with a monochromator which is fabricated from low atomic mass metal borates MxByOz crystals, wherein M is low atomic mass metal, and x, y, z are respective atom numbers of metal, borate and oxygen in chemical formula. The metal borates include borates of lithium (Li), sodium (Na) or stronium (Sr).

### BACKGROUND OF THE DISCLOSURE

### Field of the Disclosure

The present disclosure relates to X-ray optical systems. In particular, the disclosure relates to X-ray diffraction, reflection, transmission and interference optical systems fabricated from lithium (Li), sodium (Na) and strontium (Sr) borate crystals.

### Background Art Discussion

X-rays are electromagnetic radiation of exactly the same nature as light, but of much shorter wavelength. Wavelength of visible light is on the order of 6000 angstroms while the wavelength of x-rays is in the range of 0.1 to 300 angstroms. This very short wavelength is what gives x-rays their power to penetrate materials that visible light cannot. For commonly used target materials in X-ray tubes, the X-rays have well-known experimentally determined characteristic wavelengths. In addition, continuous X-ray spectra are also produced.

X-rays are classified in two different ways: Soft X-rays and Hard X-rays. The former is characterized by a relatively low energy; anything below 5 keV would be considered a soft x-ray. Soft x-rays can be absorbed in the air. The X-rays with energies above 5 keV are typically referred to as hard X-rays. Hard x-rays have the ability and energy to penetrate through different types of materials, hence, they are commonly used for industrial purposes to find internal defects in objects or parts.

X-ray technology has two primary applications: medical applications and industrial applications. The medical applications belong to two categories: diagnostic procedures, such as computer tomography (CT), fluoroscopy and others, and therapeutic procedures such as cancer treatment. The industrial applications take advantage of X-rays as an invaluable source for nondestructive radiographic testing (RT) applications providing an outlet for internal part analysis in 2D or 3D technology. For example, X-rays are a very common application of RT for accessing internal part analysis in 2D, for identifying failures or foreign material within a part. Other X-rays industrial applications include spectrometric, diffractive, reflective, interferometric and transmission testing applications providing information on composition and structure of bulk of materials and their parts as well as surface structure and topography.

X-ray optical systems include X-ray diffractometers, X-ray topography tools, extended X-ray absorption fine structure (EXAFS) and wavelength X-ray fluorescence (XRF) systems, X-ray microscopes and interferometers, as well as X-ray sources. All of these X-ray tools are based on, with rare exception, near-perfect single crystals which function as diffraction, reflection, transmission and interference optical elements. The single crystal is a solid form of substance in which atoms and molecules are arranged in a high degree of order or regular geometric periodicity throughout the entire volume of the material. The X-ray optics based on poly-crystals is also known. The poly-crystal consists of many individual single crystals, which have small sizes commonly referred as grains.

In general, there are two measuring methods employing X-ray optics: polychromatic and monochromatic. Monochromatic methods are widely used in commercial applications and, obviously, require monochromatic radiation, which is usually produced by a single-crystal monochromator. One of the characteristics common to all single-crystal monochromators is the narrow curve of reflecting intensity versus the incident angle at an angular position satisfying Bragg diffraction conditions for a given X-ray wavelength. This angular position is known as Bragg angle. The curve is referred to as a rocking curve. The width of the rocking curve is usually given as a full width at half-maximum (FWHM) value with the maximum intensity being the point at which the Bragg diffraction condition is met. For a single-crystal monochromator, typically a FWHM value does not exceed 10 20″ arcseconds. The rocking curve is also characterized by the percentage of incident radiation reflected by a crystal; this characteristic is referred to as reflectivity. The reflectivity is straightforwardly associated with the absorption of radiation in the crystal, which is determined by a linear absorption coefficient, and with a crystal structure. The latter, in turn, is characterized by a so-called structure factor. Still a further distinctive characteristic of a single-crystal X-ray monochromator is the structural perfection, i.e., the presence of a minimal amount of structural defects affecting the widening of a rocking curve and causing other undesirable effects.

FIG. 1 illustrates an example of a measured rocking curve for 400 reflection from an almost ideal silicon (Si) single crystal wafer which indicates the quality of the crystalline lattice characterized by small FWHM and relatively large reflectivity values for monochromatic Cu—K_{α1} X-ray beam irradiating the wafer. The above-mentioned 400 label of reflection refers to so-called hkl Miller indexes which designate crystal lattice planes and X-ray reflections. Based on the foregoing, for a given hkl reflection and incident X-ray wavelength, the smaller FWHM and the larger reflectivity values of the rocking curve mean the higher quality of the single-crystal X-ray monochromator quality. For an ideal single crystal of specified structure type and chemical composition, a rocking curve with the lowest FWHM and the highest reflectivity values may be calculated using the X-ray diffraction theory for a given hkl reflection and incident X-ray wavelength. This curve is referred to as an intrinsic rocking curve.

The concept of the rocking curves, measured and intrinsic, can be better understood, for example, in the context of X-ray diffractometers, which are used in a variety of applications including spectrometry, diffractometry, reflectometry, interferometry and imaging all well known to one of ordinary skill in the X-ray metrology. Each of these scientific measurement techniques uses continuous or characteristic components of the X-ray spectrum for studying the matter through its interaction with different components of the X-ray spectrum. Each technique measures results of this interaction by detecting the intensity of different components of the X-ray spectrum scattered by the irradiated sample. For a given structure and chemical composition of a sample, the factors affecting the measured intensity include the angle of incidence, angle of scattering and measurement time. These techniques are indispensable in the X-ray analysis of biological tissue, thin film analysis, sample surface and texture structure evaluation, monitoring of crystalline phase, crystal structure and lattice defects, and investigation of sample stress and strain.

Structurally, an X-ray diffractometer is configured with a crystal monochromator operating in the following manner. If an incident X-ray beam encounters the crystal lattice of the monochromator at arbitrary angle of incidence, elastic and inelastic scattering of the X-ray beam on electrons of crystal atoms occurs. Although most of the elastically scattered X-rays is eliminated due to destructive interference, when the angle of incidence equals to a specific angle (i.e. a Bragg angle), then the diffraction occurs. Some X-rays scattered in a certain direction from atomic planes are in phase with X-rays which are scattered from other atomic planes of the same kind. The scattered in-phase X-rays constructively interfere to form new enhanced wave fronts. The relation by which the diffraction occurs is known as the Bragg law or equation. Because each crystalline material has a characteristic atomic structure, it will diffract X-rays in a unique characteristic pattern.

FIG. 2 highly diagrammatically illustrates an exemplary optical schematic of X-ray diffractometer 15 including a crystal monochromator 28 diffracting X-ray radiation, which is irradiated from an X-ray source 22 and transmitted through a sample 16. The basic geometry of X-ray diffractometer 15 involves a source of polychromatic radiation 22 and an X-ray detector 24, i.e. the CCD camera indicated in this diagram, located downstream from a sample 16.

The crystal monochromator 28 is configured to ensure that the scattered or detected radiation is monochromatic. When monochromator 28 is positioned properly before or after sample 16, only the desired/selected wavelength of the X-ray spectrum emitted by an X-ray source reaches sample 16 or detector 24 after being reflected by monochromator 28 at specific angles of incidence and reflection. All other spectral wavelengths are diffracted at a slightly different angle and thus avoid detector 24. In other words, monochromator 28 operates as a spectral filter or analyzer. The X-ray intensity scattered by or transmitted through sample 16 reaches detector 24, which collects X-ray photons in time and space and transforms the collected photons into an electronic signal by a well-known signal-shaping hardware and methods related to the selected type of detector 24. The electronic signal is further processed in an electronic system known to one of ordinary skill in the art.

As discussed above, for a selected X-ray wavelength, the requirements for a high quality crystal monochromator include high reflectivity, small FWHM and low linear absorption values. These values are solely defined by structure, composition and quality (i.e. defect concentration) of the utilized crystal, as well as by the crystal's surface orientation and quality of the surface preparation. Additional requirements to be considered may be the crystal's available size and manufacturability. The adjustment of the crystal monochromator for a specific analytical method is frequently based on a tradeoff of the above-listed requirements.

There are only a few crystals used in monochromatic X-ray optics that at least partially meet the above-mentioned requirements. Among these crystals, silicon (Si) and germanium (Ge) crystals are of the highest quality (i.e. low defect concentration). Si crystals have the lower linear absorption comparing with Ge. However, Ge reflectivity is on par with Si due to larger number of electrons scattering incident X-ray radiation. The rest of the known crystals utilized for monchromators including, among others, very specific crystals with large interplanar distances, are way down on the scale of quality and size from Si and Ge crystals.

The short list of monochromator crystals becomes particularly glaring in light of ever-growing industrial demands for higher powers of X-ray radiation particularly with recently introduced synchrotrons—particle accelerators capable of producing a beam of X-rays several orders of magnitude more intense than the known conventional equipment. Moreover, some of currently used monochromatic X-ray crystals, such as acid phthalate crystals (e.g. KAP), are prone to rapidly degrade even at relatively low powers. Even the highest quality Si and Ge crystals are vulnerable to oxidation and tend to have a useful life not exceeding about 3 years. Note that Si and Ge crystal monochromators each cost about 15 20 thousand dollars not exactly a pocket change. Still other crystals, such as graphite, are known for lower quality albeit being stable and time-resistant.

A need therefore exists for utilizing in X-ray applications optics manufactured from low atomic mass number metal borates fabricated from lithium (Li), sodium (Na) and strontium (Sr) borate crystals;

Another need exists for a method of monochromatizing an X-ray radiation by utilizing the LBO crystals.

### SUMMARY OF THE DISCLOSURE

In accordance with one aspect of the disclosure, an X-ray optical system incorporates one of a refractometer, interferometer, spectrometer, diffractometer or imaging device and is configured with an X-ray source outputting an broad band X-ray radiation in a 0.01-1 nm wavelength range, and an LBO crystal-based monochromator which optically interacts with the received X-ray radiation.

In accordance with another aspect of the disclosure, a method of monochromatizing X-ray radiation includes utilizing the LBO crystal.

### DESCRIPTION OF THE DRAWINGS

Various aspects of the disclosure are discussed below with reference to the accompanying figures, which are not intended to be drawn to scale. The figures are included to provide an illustration and a further understanding of the various aspects and embodiments, and are incorporated in and constitute a part of this specification, but are not intended as a definition of the limits of any particular embodiment. The drawings, together with the remainder of the specification, serve to explain principles and operations of the described and claimed aspects and embodiments. In the figures, each identical or nearly identical component in various figures is represented by a like numeral. For purposes of clarity, not every component may be labeled in every figure. In the figures:

FIG. 1 illustrates a measured rocking curve for 400 reflection from an almost ideal silicon (Si) single crystal wafer;

FIG. 2 is an exemplary optical schematic of X-ray diffractometer of the known prior art;

FIGS. 3A 3C illustrates calculated intrinsic rocking (reflection) curves of LBO, Si and Ge, respectively;

FIG. 4 is an exemplary optical schematic of a double-crystal spectrometer with a single monochromator manufactured from an LBO crystal;

FIG. 5A 5C illustrate respective measured (experimentally obtained) rocking curves of respective LBO, Si and Ge.

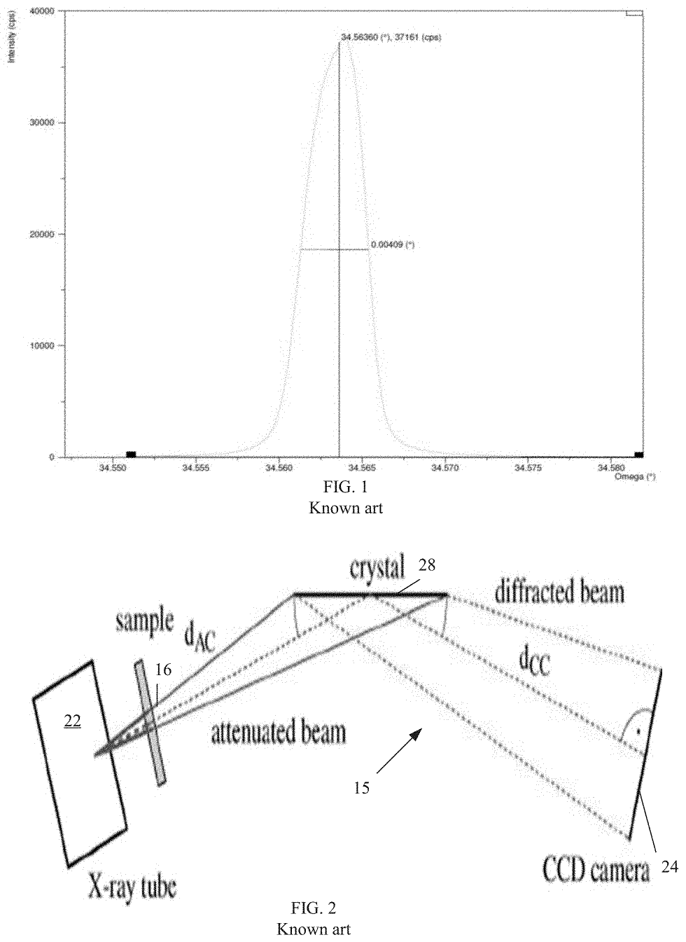

### SPECIFIC DESCRIPTION

Described herein are optical schematics of X-ray diffractometers used in X-ray spectrometry, diffractometry, reflectometry, interferometry and imaging. In particular, the shown schematics each are include a monochromator configured in LBO crystals and operating in a reflective or transmissive mode. The LBO monochromator offers several advantages, including a narrow rocking curve, high reflectivity and high mechanical integrity.

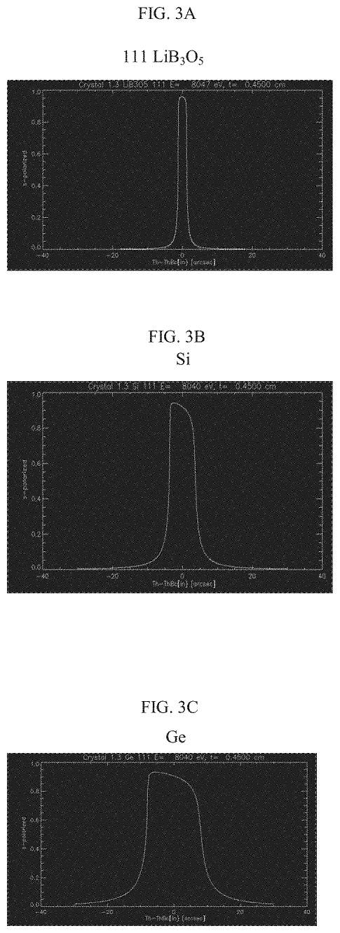

FIG. 3A 3C illustrate respective calculated intrinsic rocking (reflection) curves, in relative units, i.e. intensities reflected from atomic planes versus the angle of incidence of a monochromatic X-ray beam. In particular, the curves are calculated for strongest symmetric 111 reflection of CuKa₁ X-ray in Bragg geometry for respective single crystal plates of LBO (FIG. 3A), Si (FIG. 3B) and Ge (FIG. 3C). In symmetrical Bragg geometry, reflecting atomic planes, such as (111), are parallel to the upstream surface of the monochromator or a sample to be tested. As can be seen, the intrinsic rocking curve of LBO has a FWHM, which is almost three times less than that of Si, and almost 6 times less than that of Ge. The theoretical peak reflectivity and linear absorption parameters of the LBO are also better than those of respective Si and Ge as summarized in the following table.

TABLE 1
Parameters for the theoretical crystal
intrinsic rocking (reflection) curves.

| | LiB₃O₅ | Si | Ge |
|---|---|---|---|
| Single crystals | | | |
| Reflecting crystallographic plane (hkl) | (111) | | |
| Energy and wavelength for incident Cu- | 8.0478 keV and 0.15406 nm | | |
| Ka₁ radiation | | | |
| Calculated Bragg angle Theta, deg of arc | 11.77 | 14.22 | 13.64 |
| Parameters of calculated intrinsic | | | |
| reflection curves | | | |
| FWHM, arcsec | 2.53 | 7.6 | 16.7 |
| Peak reflectivity, relative units | 0.96 | 0.94 | 0.93 |
| Linear absorption coefficient, cm⁻¹ | 20 | 141 | 353 |

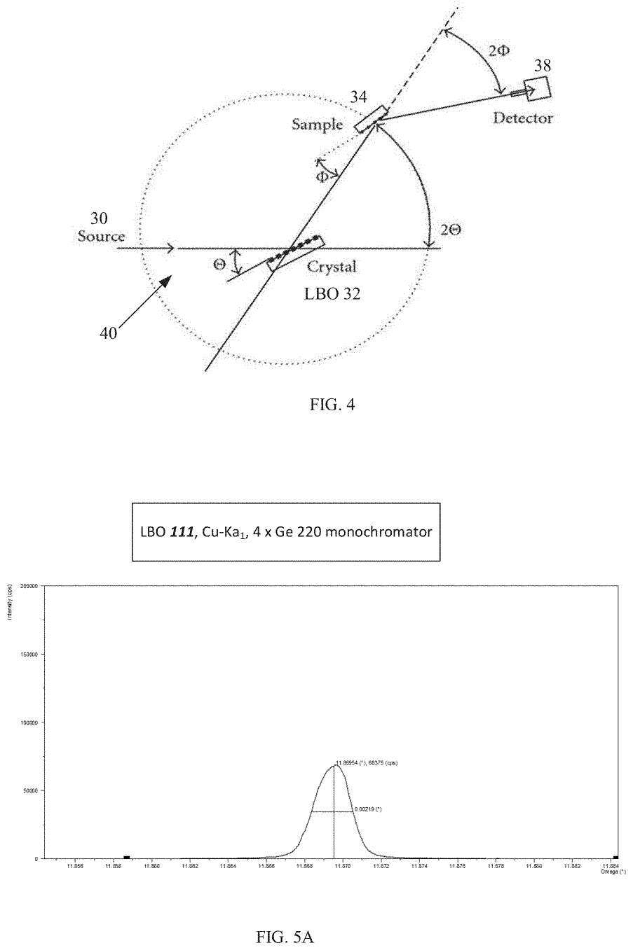

FIG. 4 illustrates an exemplary optical schematic of a single-crystal X-ray spectrometer 40. The spectrometer 40 includes an X-ray source 30 selected from conventional tubes, rotation anode systems and synchrotrons. While the scope of the invention includes all of the above-mentioned types of X-ray source 30, preferably, the source is a hard energy source emitting hard X-rays, but the latter does not exclude the possibility of working with soft X-rays. The polychromatic X-ray radiation is incident on a monochromator 32 at an angle of incidence Θ.

In accordance with a main concept of the invention, monochromator 32 is made of borates of lithium (LiB₃O₅) or strontium (SrB₄O₇) or sodium borates. A material for a monochromator can be selected single-crystal or polycrystalline. For the purpose of convenience, this description further refers to LBO single crystal, but the entire disclosure relates to a group of borates of low atomic mass metals including additional compounds each having different chemical formulas. For example, LBO besides LiB₃O₅ may include LiBO₂ and Li₂B₄O₇. Thus, for the purposes of generalization, the metal borates covered in this disclosure are referred to as MₓB_{y}O_{z}, wherein M is Li, Na and Sr, and x, y. z are numbers of atoms in a chemical formula of a compound.

The monochromator 32 is a reflector which selects a narrow spectral band of broadband X-ray beam from source 30 and reflects this intense monochromatic beam on a single-crystal sample 34. The angle of incidence equals to the reflection angle at reflecting plane of monochromator 32, so that the shown diffraction schematic of monochromator is symmetric. The angle of incidence Θ at reflecting plane of monochromator 32 equals to or it is close to an angle of incidence Φ at receiving/upstream reflecting plane of single-crystal sample 34, so that the shown diffraction schematic is called non-dispersive. However, sample 34 may represent not only single crystals but also polycrystalline materials, liquids and even gases; for analysis of these samples, a wide range of angles of incidence is utilized. Thus, the monochromatic X-ray beam irradiates single-crystal sample 34 at incidence angle Φ; the sample 34 reflects the incident beam at the same angle. A detector 38 is set at an angle 2Φ relative to incident beam position to collect X-ray photons reflected from the single-crystal sample 34.

A variation of the optical schematic of FIG. 4 may include a triple-crystal X-ray spectrometer in symmetric diffraction scheme. Specifically, this scheme includes monochromator, such as LBO or borates of sodium (Na) or strontium (Sr), receiving a polychromatic beam of X-rays from the X-ray source. The monochromator reflects the desired monochromatic beam which is incident on the sample to be examined similarly to the schematic of FIG. 4. The monochromatic beam reflected from the sample is further incident on an analyzer crystal, which is identical to the monochromator. The analyzer reflects the received X-rays onto the detector. The use of the analyzer provides background reduction, as well as improving resolution of rocking curves collected for the sample.

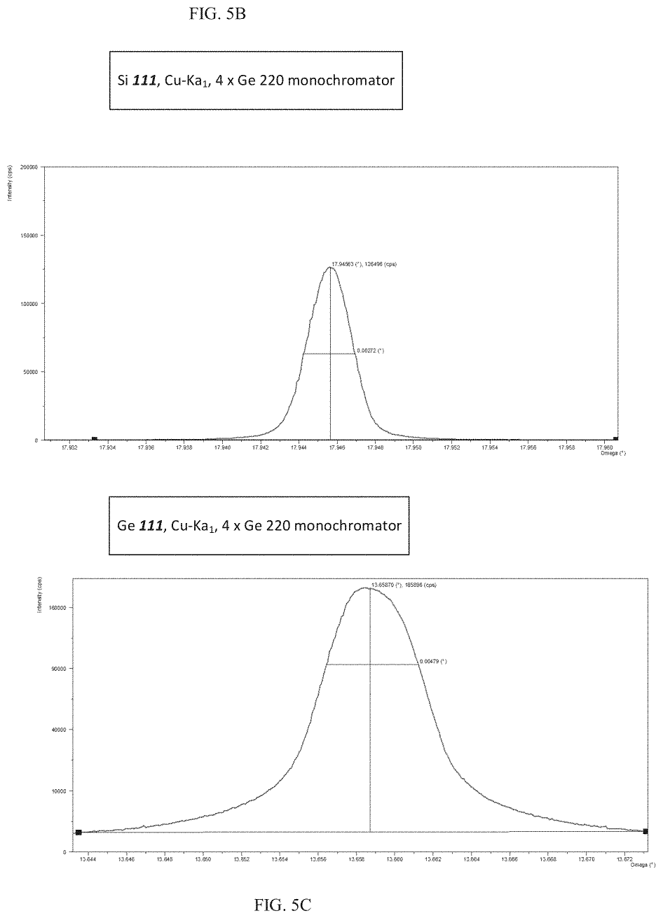

FIGS. 5A 5C illustrate respective rocking curves for strongest, 111 reflections measured in count per second with changing angle of incidence of the monochromatic radiation. In particular, the experiments were conducted on −0.7 mm thick, flat LBO, Si and Ge crystal plates in symmetrical Bragg geometry with monochromatic Cu—Ka₁ X-rays. Parameters of these rocking curves are shown in table 2.

TABLE 2
Parameters of measured 111 reflection
curves displayed at FIG. 6A-6C.

| | Measured values, Cu-Ka₁, | | |
|---|---|---|---|
| | 4x Ge 220 monochromator | | |
| | | Peak max | Peak integral |
| Reflection | FWHM, | intensity, | intensity, |
| curve | sec | cps | cps |
| LBO 111 | 7.9 | 68400 | 165 |
| Si 111 | 9.8 | 126500 | 376 |
| Ge 111 | 17.2 | 185900 | 983 |

The values of the measured FWHM and peak integral intensity change among LBO, Si and Ge in line with a change of respective values calculated for intrinsic reflection curves shown in Table 1 and FIGS. 3A 3C. Observed absolute differences between measured and calculated FWHM values for each crystal are explained by optical aberrations related to Ge 220 monochromator and limit of minimal angular step specific to utilized model of X-ray diffractometer. However, the measured peak maximum intensity for LBO is lower than that of the calculated reflection curve. This is explained by uncertainties in calculations of atomic scattering factor for Li and temperature factors for Li, B and O atoms in LBO crystal, and also due to the experimental nature of the measured LBO crystal plate with (111) orientation, which is unusual for this material.

Peak maximum intensity of LBO 111 reflection may be increased 1.5-2.6 times by asymmetric Bragg diffraction, i.e. a reflection of X-rays from (111) atomic planes which are not parallel to the surface of LBO crystal plate. For this purpose, as an example, the monochromator 32 at FIG. 4 is intentionally cut from LBO crystal so that its reflecting (111) atomic planes create an angle with the surface of the monochromator plate; this angle is slightly less than Bragg angle for 111 reflection, thus minimizing an angle of incidence relative to crystal surface. This type of monochromator is referred to as the asymmetric monochromator.

Having thus described several aspects of at least one example, it is to be appreciated that various alterations, modifications, and improvements will readily occur to those skilled in the art. For example, the sample to be analyzed may located upstream from the monochromator. Such alterations, modifications, and improvements are intended to be part of this disclosure, and are intended to be within the scope of the examples discussed herein. Accordingly, the foregoing description and drawings are by way of example only.

1. An X-ray optical system, which incorporates a refractometer, interferometer, spectrometer, diffractometer or imaging device for analyzing a sample, comprising a monochromator fabricated from a group of low atomic mass metal borates MxByOz, wherein M is low atomic mass metal, and x, y, z are respective atom numbers of metal, borate and oxygen.

2. The X-ray optical system of claim 1, wherein the low mass-metal is one of lithium (Li), sodium (Na) or stronium (Sr).

3. The X-ray optical system of claim 1, wherein the x, y and z atomic numbers vary in accordance with a desired chemical formula of the selected metal borate.

4. The X-ray optical system of claim 1, wherein the monochromator operates in a reflective mode or transmissive mode.

5. The X-ray optical system of claim 1, wherein the monochromator is a single crystal or polycrystal.

6. The X-ray optical system of claim 1 further comprising:
an X-ray source emitting a polychromatic X-ray beam which is incident on the monochromator reflecting a filtered monochromatic beam; and
a X-ray detector spaced upstream from the sample and detecting the monochromatic beam.

7. The X-ray optical system of claim 6, wherein the monochromator is located upstream or downstream from a sample.

8. The X-ray optical system of claim 6, wherein the X-ray source is selected from conventional tubes, rotation anode systems, or synchrotron.

9. The X-ray optical system of claim 7, wherein the sample is one of solid, gas or liquid.

10. The X-ray system of claim 6 further comprising an analyzer configured identically to the monochromator and located immediately upstream from the detector.

11. A method of monochromatizing X-ray radiation, comprising:
emitting a polychromatic beam of X-ray radiation along a path; and
spectrally and spatially filtering the polychromatic beam by a monochromator selected from low atomic mass number metal borates, thereby forming a monochromatic beam.

12. The method of claim 11 further comprising detecting the monochromatic beam.

13. The method of claim 11, wherein the metal borates include Li, Na or Sr borates.

14. The method of claim 11 further comprising locating the monochromator along the path upstream or downstream from a sample to be analyzed.

15. The method of claim 11, wherein the monochromator operates in a reflective mode or transmissive mode.
